(19) United States
(12) Patent Application Publication
(10) Pub. No.: US 2024/0090843 A1
(43) Pub. Date: Mar. 21, 2024

(54) PHYSIOLOGICAL INFORMATION PROCESSING METHOD AND PHYSIOLOGICAL INFORMATION PROCESSING APPARATUS

(71) Applicant: NIHON KOHDEN CORPORATION, Tokyo (JP)

(72) Inventors: Yoshiaki NAKAO, Tokorozawa-shi (JP); Toshiyuki TAURA, Tokorozawa-shi (JP); Shumpei YANO, Tokorozawa-shi (JP)

(21) Appl. No.: 18/460,222

(22) Filed: Sep. 1, 2023

(30) Foreign Application Priority Data
Sep. 16, 2022 (JP) 2022-148335

(51) Int. Cl. A61B 5/00; A61B 5/369

(52) U.S. Cl. CPC A61B 5/7203; A61B 5/369; A61B 5/7221; A61B 5/742

(57) ABSTRACT

A physiological information processing method executed by a computer. The physiological information processing method includes obtaining a plurality of electroencephalogram signals indicating an electroencephalogram of a subject, using a plurality of electrodes attached to a head of the subject, each of the plurality of electroencephalogram signals being associated with a respective one of the plurality of electrodes, obtaining, from each of the plurality of electroencephalogram signals, a plurality of artifact component signals indicating different types of artifacts, calculating effective values of the plurality of artifact component signals of each of the electroencephalogram signals, determining whether the effective value of each of the artifact component signals of the electroencephalogram signal satisfies a predetermined condition, and visually presenting information related to the artifact associated with the artifact component signal of which the effective value satisfies the predetermined condition.

### CROSS-REFERENCE TO RELATED APPLICATIONS

This application is based on and claims priority under 35 USC 119 from Japanese Patent Application No. 2022-148335 filed on Sep. 16, 2022, the entire content of which is incorporated herein by reference.

### TECHNICAL FIELD

The present disclosure relates to a physiological information processing method and a physiological information processing apparatus. In particular, the present disclosure relates to a physiological information processing method and a physiological information processing apparatus in which information related to an artifact mixed in an electroencephalogram signal is visually presented. Further, the present disclosure further relates to a program that causes a computer to execute the information processing method, and a non-transitory computer-readable storage medium storing the program.

### BACKGROUND ART

An electroencephalograph configured to noninvasively measures an electroencephalogram indicating an electrical activity of brain neurons of a patient is known. In the electroencephalograph, electroencephalogram signals are obtained through a plurality of electrodes attached to a head of the patient. On the other hand, since the electroencephalogram signals are weak electrical signals of a living body which indicate the electrical activity of the brain neurons, artifacts, which are noises other than the electroencephalogram, are likely to be mixed in the electroencephalogram signals. U.S. Pat. No. 9,055,927B discloses a technique for detecting, separating, and reducing various artifacts mixed in electroencephalogram signals of respective channels. Further, U.S. Pat. No. 9,055,927B discloses a technique of indicating a type of each of the artifacts and re-extracting an electroencephalogram component signal according to the type of artifact to be reconstructed.

At this time, in the electroencephalogram signals from which various artifacts have been reduced by software through a signal process of reducing the artifacts, waveform distortion may occur compared to original electroencephalogram signals before an artifact is reduced. As described above, reliability of the electroencephalogram signals from which the artifacts are reduced through the signal process is lower than reliability of electroencephalogram signals in which the artifacts are not mixed. Therefore, in order to record electroencephalogram signals having a high signal quality, it is essential to eliminate factors of the artifacts when electroencephalogram measurement is started.

On the other hand, the artifacts mixed in the electroencephalogram include various types of artifacts such as artifacts caused by a living body (such as electro cardiogram and eye movement) and artifacts caused by factors other than the living body (such as a contact failure of an electrode). Further, measures for reducing various artifact differ from each other. For this reason, a medical worker who has little experience and is unfamiliar with the artifact reduction measure may find it difficult to record an electroencephalogram signal having a signal quality that is less contaminated with the artifact. From the above viewpoint, there is room for consideration of a user interface to support recording of the electroencephalogram signal having a signal quality that is less contaminated with the artifact.

### SUMMARY OF INVENTION

Aspect of non-limiting embodiments of the present disclosure relates to provide a physiological information processing method and a physiological information processing apparatus that support recording of an electroencephalogram signal having a high signal quality, and relates to provide a program that causes a computer to execute the information processing method, and a computer readable storage medium storing the program.

Aspects of certain non-limiting embodiments of the present disclosure address the features discussed above and/or other features not described above. However, aspects of the non-limiting embodiments are not required to address the above features, and aspects of the non-limiting embodiments of the present disclosure may not address features described above.

According to an aspect of the present disclosure, there is provided a physiological information processing method executed by a computer, the physiological information processing method including:
obtaining a plurality of electroencephalogram signals indicating an electroencephalogram of a subject, using a plurality of electrodes attached to a head of the subject, each of the plurality of electroencephalogram signals being associated with a respective one of the plurality of electrodes;
obtaining, from each of the plurality of electroencephalogram signals, a plurality of artifact component signals indicating different types of artifacts;
calculating effective values of the plurality of artifact component signals of each of the electroencephalogram signals;
determining whether the effective value of each of the artifact component signals of the electroencephalogram signal satisfies a predetermined condition; and
visually presenting information related to the artifact associated with the artifact component signal of which the effective value satisfies the predetermined condition.

According to an aspect of the present disclosure, there is provided a physiological information processing apparatus including:
one or more processors; and
one or more memories configured to store a computer readable instruction,
in which in a case where the computer readable instruction is executed by the one or more processors, the physiological information processing apparatus is configured to:
   obtain a plurality of electroencephalogram signals indicating an electroencephalogram of a subject, using a plurality of electrodes attached to a head of the subject, each of the plurality of electroencephalogram signals being associated with a respective one of the plurality of electrodes,
   obtain, from each of the plurality of electroencephalogram signals, a plurality of artifact component signals indicating different types of artifacts,
   calculate effective values of the plurality of artifact component signals of each of the electroencephalogram signals,
   determine whether the effective value of each of the artifact component signals of the electroencephalogram signal satisfies a predetermined condition, and
   visually present information related to the artifact associated with the artifact component signal of which the effective value satisfies the predetermined condition.

### BRIEF DESCRIPTION OF DRAWINGS

Exemplary embodiment(s) of the present invention will be described in detail based on the following figures, wherein:

FIG. 1 is a block diagram illustrating a configuration of a physiological information processing apparatus according to an exemplary embodiment (hereinafter referred to as a present embodiment) of the presently disclosed subject matter;

FIG. 2 is a schematic diagram illustrating a plurality of electrodes attached to a head of a patient;

FIG. 3 is a flowchart illustrating a physiological information processing method according to the present embodiment;

FIG. 4 is a diagram illustrating an example of electroencephalogram signals obtained from the electrodes attached to the patient, and source waveforms obtained through blind source separation;

FIG. 5 is a diagram illustrating that an electroencephalogram signal XFp1 of an electrode Fp1 includes an electroencephalogram component signal B and a plurality of artifact component signals C1 to C5;

FIG. 6 is a diagram illustrating an example of an electroencephalogram display screen on which electroencephalograms of channels are displayed;

FIG. 7 is a diagram illustrating an example of artifact related information for each type of artifact; and

FIG. 8 is a diagram illustrating an example of an electrode display screen on which an electrode image illustrating a type of artifact mixed in the electroencephalogram signal of each of the electrodes is displayed.

### DESCRIPTION OF EMBODIMENTS

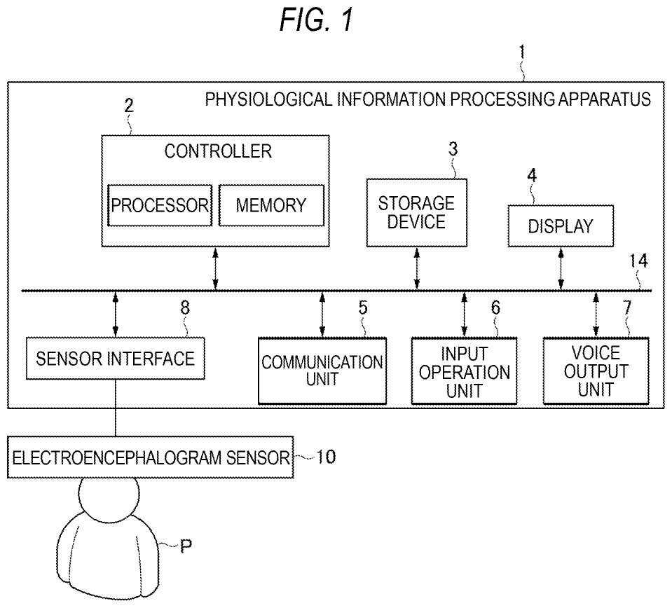

Hereinafter, the present embodiment will be described with reference to the drawings. FIG. 1 is a block diagram illustrating a configuration of a physiological information processing apparatus 1 according to the present embodiment. As illustrated in FIG. 1, the physiological information processing apparatus 1 (hereinafter, simply referred to as a processing apparatus 1) may include a controller 2, a storage device 3, a display 4, a communication unit 5, an input operation unit 6, a voice output unit 7, and a sensor interface 8. These components provided in the processing apparatus 1 are communicably connected to one another via a bus 14.

The processing apparatus 1 may be a medical device (for example, a patient monitor or an electroencephalograph) configured to display physiological information of a subject P (patient), a personal computer, a workstation, a smartphone, a tablet, or a wearable device (for example, an AR glass) worn on a body (for example, an arm or a head) of a medical worker. The processing apparatus 1 is configured to output information indicating electroencephalogram of the subject P.

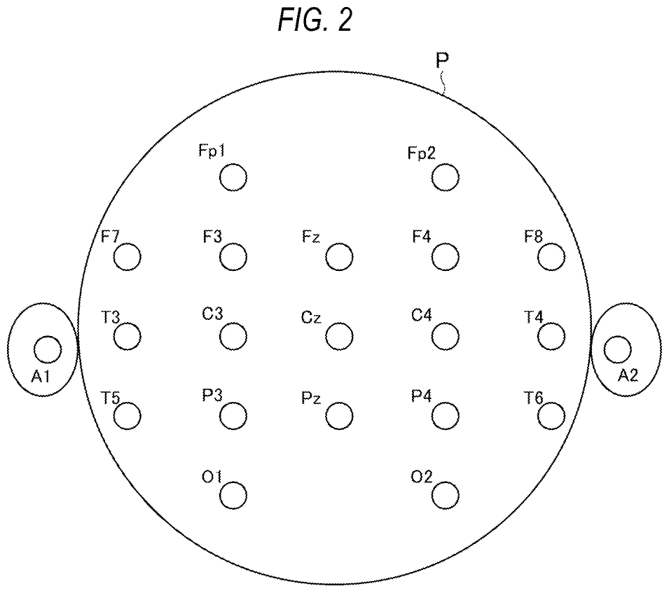

The controller 2 may include one or more memories and one or more processors. Each memory is configured to store a computer readable instruction (program). For example, the memory may include a read only memory (ROM) in which various programs are stored, a random access memory (RAM) having a plurality of work areas in which various programs executed by the one or more processors are stored, and the like. Each processor may include at least one of, for example, a central processing unit (CPU), a micro processing unit (MPU), and a graphics processing unit (GPU). The CPU may include a plurality of CPU cores. The GPU may include a plurality of GPU cores. The processor may be configured to load a program designated from various programs incorporated in the storage device 3 or ROM into RAM, and configured to execute various processes in cooperation with the RAM. In particular, the processor is configured to load a physiological information processing program for executing a series of processes illustrated in FIG. 2 into the RAM, and is configured to execute the program in cooperation with the RAM. Details of the physiological information processing program will be described later.

The storage device 3 is a storage device (storage) such as a hard disk drive (HDD), a solid state drive (SSD), or a flash memory, and stores programs and various data. The physiological information processing program may be incorporated in the storage device 3. Further, the storage device 3 may store electroencephalogram data of the subject P. For example, the electroencephalogram data (electroencephalogram signals) obtained from an electroencephalogram sensor 10 may be stored in the storage device 3 via the sensor interface 8.

The communication unit 5 is configured to connect the processing apparatus 1 to an in-hospital network. Specifically, the communication unit 5 may include various wired connection terminals configured to communicate with a central monitor or a server disposed in the in-hospital network. Further, the communication unit 5 may include a wireless communication module configured to wirelessly communicate with the central monitor or the server. The communication unit 5 may include, for example, a wireless communication module corresponding to a medical telemetry system. The communication unit 5 may include a wireless communication module corresponding to a wireless communication standard such as Wi-Fi (registered trademark) or Bluetooth (registered trademark) and/or a wireless communication module corresponding to a mobile communication system using SIM. The in-hospital network may include, for example, a local area network (LAN) or a wide area network (WAN). The processing apparatus 1 may be connected to Internet via the in-hospital network.

The display 4 is configured to display the physiological information (information related to the electroencephalogram) of the subject P. The display 4 can include, for example, a liquid crystal panel or an organic EL panel. The input operation unit 6 is, for example, a touch panel, a mouse, and/or a keyboard, which are arranged over the display 4. The input operation unit 6 is configured to receive an input operation of the medical worker and configured to generate an operation signal corresponding to the input operation of the medical worker. After the operation signal generated by the input operation unit 6 is transmitted to the controller 2 via the bus 14, the controller 2 is configured to execute a predetermined operation according to the operation signal. The voice output unit 7 can include one or more speakers.

The sensor interface 8 is an interface configured to connect the electroencephalogram sensor 10 to the processing apparatus 1. The sensor interface 8 may include an input terminal to which the electroencephalogram signals output from the electroencephalogram sensor 10 are input. The electroencephalogram sensor 10 is configured to noninvasively measure the electroencephalogram signals indicating an electrical activity of brain neurons of the subject P. The electroencephalogram sensor 10 can include a plurality of electrodes attached to a head of the subject P.

As illustrated in FIG. 2, for example, the electroencephalogram sensor 10 can include 21 electrodes mounted on the head or near the head of the subject P. Specifically, an electrode A1 is attached to a left earlobe. An electrode A2 is attached to a right earlobe. An electrode Fz is attached to a frontal portion of a median line connecting a root of a nose and an external occipital protuberance. An electrode Cz is attached to a center of the median line. An electrode Pz is attached to a parietal region of the median line. Electrodes Fp1 and Fp2 are attached to a front section. Electrodes F3 and F4 are attached to the frontal portion. Electrodes C3 and C4 are attached to a central portion. Electrodes P3 and P4 are attached to the parietal region. Electrodes O1 and O2 are attached to an occipital region. An electrode F7 is attached to a front side of a left head portion. An electrode T3 is attached to a center of the left head portion. An electrode T5 is attached to a rear side of the left head portion. An electrode F8 is attached to a front side of a right head portion. An electrode T4 is attached to a center of the right head portion. An electrode T6 is attached to a rear side of the right head portion.

As a method of synthesizing the electroencephalogram, a homopolar lead method for recording a potential difference between potential of a head and potential of an earlobe, a bipolar lead method for recording a potential difference of the head between two points, or an average reference electrode method is applied.

The sensor interface 8 may include at least a plurality of amplifier circuits and an AD converter. Each of the plurality of amplifier circuits is configured to amplify the electroencephalogram signal output from the corresponding electrode. The AD converter is configured to convert the electroencephalogram signal from an analog signal to a digital signal. The electroencephalogram signal converted into the digital signal is transmitted from the sensor interface 8 to the controller 2.

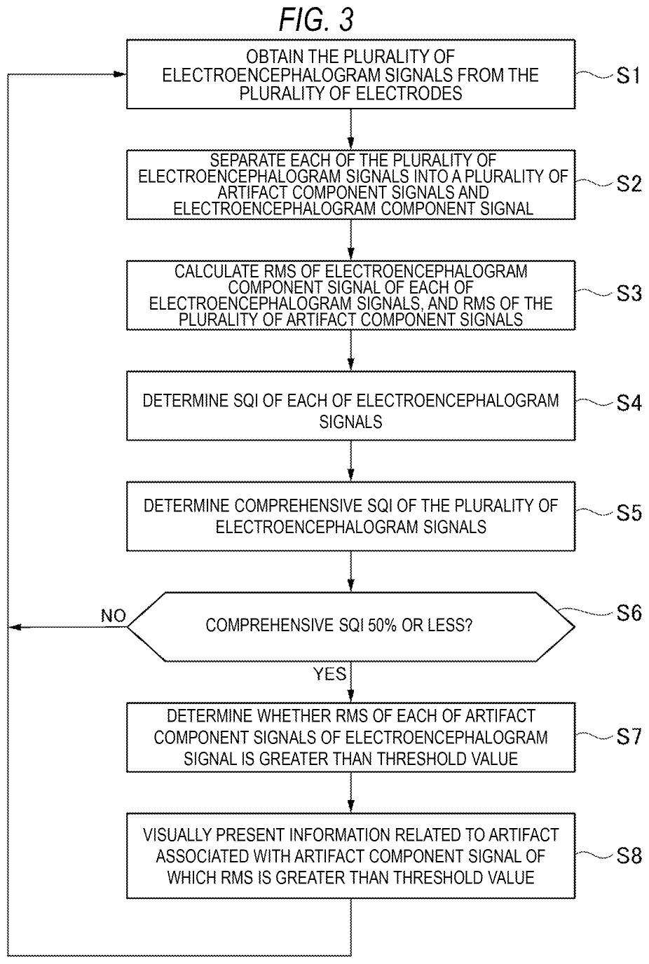

Next, the physiological information processing method according to the present embodiment will be described below with reference to FIG. 3. FIG. 3 is a flowchart illustrating the physiological information processing method according to the present embodiment. Processing illustrated in FIG. 3 are repeatedly executed at predetermined intervals (for example, every two seconds).

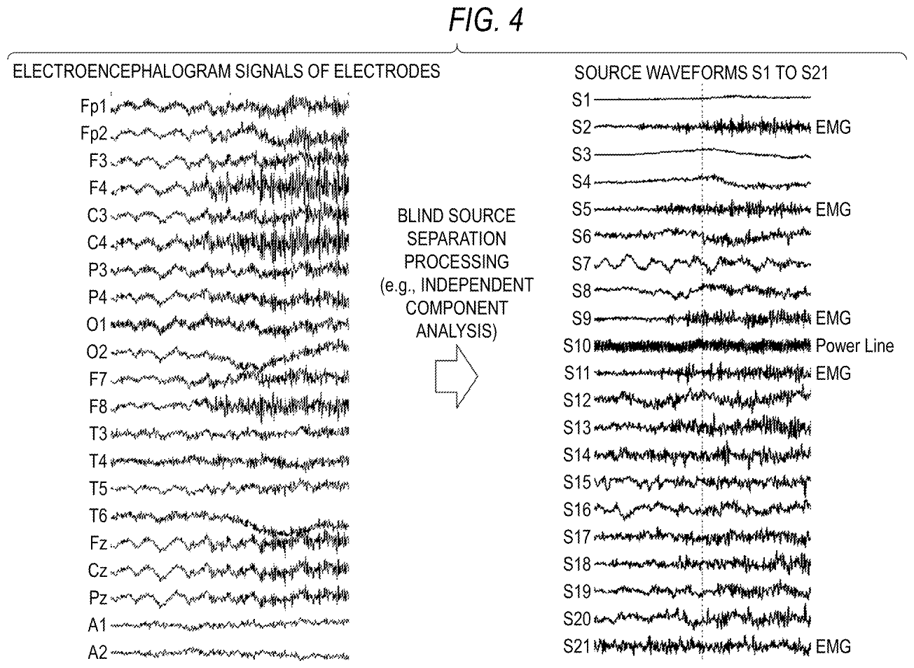

As illustrated in FIG. 3, in step S1, the controller 2 is configured to obtain, via the sensor interface 8, the plurality of electroencephalogram signals (digital signals) indicating the electroencephalogram of the subject P, from the electroencephalogram sensor 10 including the plurality of electrodes attached to the head of the subject P. In the present embodiment, 21 types of electroencephalogram signals are obtained from 21 electrodes attached to the head of the subject P. That is, each of the electroencephalogram signals is associated with a respective one of the 21 electrodes. As illustrated in FIG. 4, a first electroencephalogram signal shown at the top among the 21 types of electroencephalogram signals indicates an electroencephalogram signal obtained from the electrode Fp1. A second electroencephalogram signal from the top indicates an electroencephalogram signal obtained from the electrode Fp2. In step S1, the electroencephalogram signals for a predetermined period (for example, a two-second epoch) are acquired.

Next, the controller 2 is configured to separate, into a plurality of artifact component signals and an electroencephalogram component signal, each of the plurality of electroencephalogram signals (step S2 in FIG. 3). In the present embodiment, there is a high probability that different types of artifacts are mixed in the electroencephalogram signal of each electrode. Therefore, each electroencephalogram signal includes the electroencephalogram component signal indicating a component the electroencephalogram, and the plurality of artifact component signals indicating a plurality of artifacts indicating different types of artifacts. The controller 2 is configured to separate each electroencephalogram signal into the electroencephalogram component signal and the artifact component signals, through a blind source separation (BSS) processing such as an independent component analysis (ICA).

The independent component analysis is a calculation method for separating a multivariate signal into a plurality of additive components. In the present embodiment, since the 21 types of electroencephalogram signals are simultaneously obtained by 21 electrodes, the 21 types of electroencephalogram signals can be decomposed into components of 21 types of source waveforms S₁ to S₂₁ by the independent component analysis (see FIG. 4).

In a case where the 21 types of electroencephalogram signals X(t) are set to X(t)=(X_{Fp1}(t), . . . , X_{A2}(t))^{T}, and the 21 types of source waveforms S(t) are (S₁(t), . . . , S₂₁(t))^{T}, a relationship between X(t) and S(t) in the independent component analysis is expressed as X(t)=H·S(t). Here, H is a mixing matrix of 21 rows×21 columns. Specifically, the relationship between X(t) and S(t) is expressed by the following formula (1).

$$\\ \begin{matrix}[ \begin{matrix}{X}_{{F}_{p 1}} \\ {X}_{{F}_{p 2}} \\ ⋯ \\ {X}_{Λ 2}\end{matrix} ] = [ \begin{matrix}{h}_{11} & {h}_{12} & ⋯ & {h}_{121} \\ {h}_{21} & {h}_{22} & ⋯ & {h}_{221} \\ ⋯ & ⋯ & ⋯ & ⋯ \\ {h}_{211} & {h}_{212} & ⋯ & {h}_{2121}\end{matrix} ] [ \begin{matrix}S 1 \\ S 2 \\ ⋯ \\ S 21\end{matrix} ] & ( 1 )\end{matrix}$$

Thus, the controller 2 can express the electroencephalogram signal X(t) as H·S(t), through the independent component analysis. That is, each of the electroencephalogram signals X_{Fp1} to X_{A2} can be expressed by 21 types of source signals S₁ to S₂₁. Here, the electroencephalogram signal X_{Fp1} of the electrode Fp1 can be expressed as X_{Fp1}=h_{1 1}S₁+h_{1 2}S₂+ . . . h_{1 21}S₂₁. The electroencephalogram signal X_{A2} of the electrode A2 can be expressed as X_{A2}=h_{21 1}S₁+h_{21 2}S₂+ . . . h_{21 21}S₂₁. In the following description, the electroencephalogram signals may be collectively referred to simply as electroencephalogram signals X.

Next, the controller 2 is configured to classify the 21 types of source waveforms S₁ to S₂₁. In the present embodiment, the source waveform includes a source waveform indicating the electroencephalogram and a source waveform indicating the artifact. The source waveform indicating the artifact includes a source waveform indicating an artifact caused by a living body of the subject P, and a source waveform indicating an artifact caused by a factor other than the living body. The source waveform indicating the artifact caused by the living body includes a source waveform indicating an artifact caused by an electro cardiogram of the subject P, a source waveform indicating an artifact caused by eye movement of the subject P, and a source waveform indicating an artifact caused by an electromyogram of the subject P. On the other hand, the source waveform indicating the artifact caused by the factor other than the living body includes a source waveform indicating an artifact caused by an attachment failure of the electrode, and a source waveform indicating an artifact caused by an electrode lead wire.

As described above, the source waveforms S₁ to S₂₁ are classified into any one of i) the source waveform indicating the electroencephalogram, ii) the source waveform indicating the artifact caused by the electro cardiogram, iii) the source waveform indicating the artifact caused by the eye movement, iv) the source waveform indicating the artifact caused by the electromyogram, v) the source waveform indicating the artifact caused by the attachment failure of the electrode, and vi) the source waveform indicating the artifact caused by the electrode lead wire. As an example of a method for classifying the source waveform, a type of the source waveform may be determined based on a correlation coefficient between the source waveform and each of reference waveforms related to various artifacts. For example, in a case where the correlation coefficient between a source waveform S₁₀ and a reference waveform indicating the artifact caused by the electrode lead wire is high, the source waveform S₁₀ may be classified as the source waveform indicating the artifact caused by the electrode lead wire. In this manner, types of the artifacts indicated by the source waveforms are identified based on correlation coefficients between the source waveforms and the reference waveforms for the various artifacts. Further, in a case where a predetermined source waveform does not correlate with any type of artifact-related reference waveform, the predetermined source waveform may be classified as the source waveform indicating the electroencephalogram.

For details of the method for classifying each of the source waveforms, reference is made to the aforementioned U.S. Pat. No. 9,055,927B and the following non-patent literature. The following non-patent literature describes an example of a method for classifying the source waveform in detail. Non-patent Literature: Wallstrom G L, Kass R E, Miller A, Cohn J F, Fox N A (2004). Automatic correction of ocular artifacts in the EEG: a comparison of regression-based and component-based methods. Int J Psychophysiol 53: 105-119.

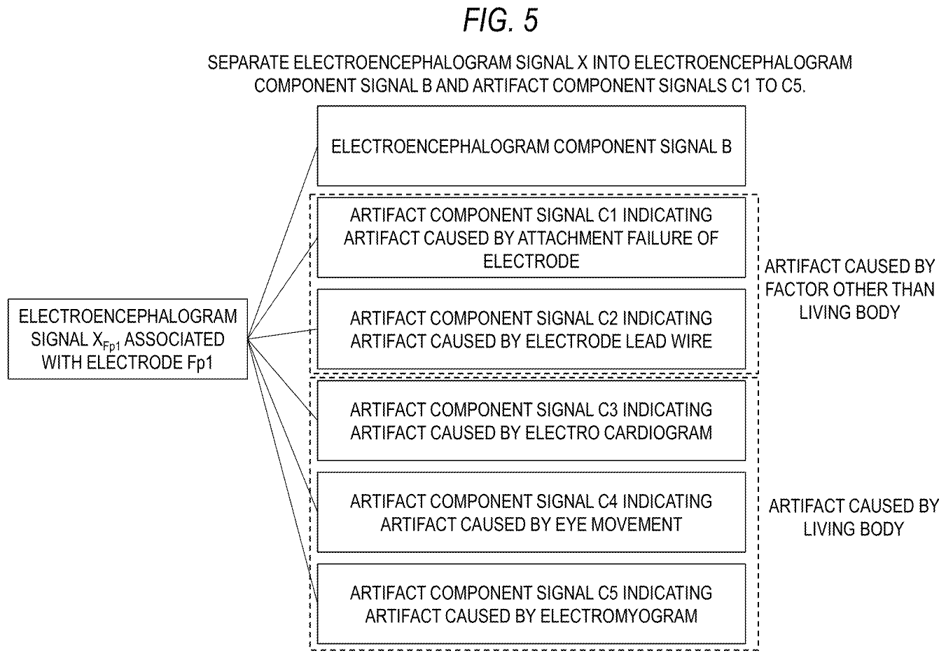

As described above, after types of the source waveforms S₁ to S₂₁ are specified, the controller 2 is configured to separate each of the electroencephalogram signals X into the electroencephalogram component signal B indicating the component of the electroencephalogram and the artifact component signals C1 to C5. As illustrated in FIG. 5, the artifact component signal C1 is an artifact component signal indicating the artifact caused by the attachment failure of the electrode. The artifact component signal C2 is an artifact component signal indicating the artifact caused by the electrode lead wire. The artifact component signal C3 is an artifact component signal indicating the artifact caused by the electro cardiogram of the subject P. The artifact component signal C4 is an artifact component signal indicating the artifact caused by the eye movement of the subject P. The artifact component signal C5 is an artifact component signal indicating the artifact caused by the electromyogram of the subject P.

As illustrated in FIG. 5, the electroencephalogram signal X_{Fp1} associated with the electrode Fp1 is separated into the electroencephalogram component signal B and the artifact component signals C1 to C5. Each of the electroencephalogram signals X other than the electroencephalogram signal X_{Fp1} is separated into the electroencephalogram component signal B and the artifact component signals C1 to C5.

For example, as illustrated in FIG. 4, in a case where the source waveforms S2, S5, S9, S11, and S21 are source waveforms indicating artifacts caused by the electromyogram, the artifact component signal C5 of the electroencephalogram signal X_{Fp1} is calculated as C5_{Fp1}=h_{1 2}S₂+h_{1 5}S₅+h_{1 9}S₉+h_{1 11}S₁₁+h_{1 21}S₂₁. In a case where the source waveform S₁₀ is a source waveform indicating the artifact caused by the electrode lead wire, the electroencephalogram component signal C2 of the electroencephalogram signal X_{Fp1} is calculated as C2_{Fp1}=h_{1 10}S₁₀. Furthermore, the artifact component signal C5 of the electroencephalogram signal X_{A2} is calculated as C5_{A2}=h_{21 2}S₂+h_{21 5}S₅+h_{21 9}S₉+h_{21 11}S₁₁+h_{21 21}S₂₁. In this way, each of the electroencephalogram signals X is separated into the electroencephalogram component signal B and the artifact component signals C1 to C5, through the independent component analysis.

Referring back to FIG. 3, the controller 2 is configured to calculate a root mean square (RMS) of each of the electroencephalogram signals X (more specifically, the electroencephalogram signals X_{Fp1} to X_{A2}), RMS of the electroencephalogram component signal B of each of the electroencephalogram signals X, and RMS of the artifact component signals C1 to C5 of the electroencephalogram signals X (step S3). In the present embodiment, since the electroencephalogram signals X in the predetermined period (for example, a 2-second epoch) are obtained from the electrodes Fp1 to A2 in step S1, RMS of each of the electroencephalogram signals X, RMS of the electroencephalogram component signal B of each of the electroencephalogram signals X, and RMS of the artifact component signals C1 to C5 of each of the electroencephalogram signals X in the predetermined period are calculated. Further, in the present embodiment, RMS of each component signal is calculated as an example of an effective value, but the effective value (index value) other than RMS may be calculated.

In step S4, the controller 2 is configured to determine a signal quality index (SQI) of each of the electroencephalogram signals X_{Fp1} to X_{A2}. The SQI is an index indicating a signal quality of the electroencephalogram signal. In this respect, the controller 2 is configured to determine SQI of each of the electroencephalogram signals X, based on RMS of the electroencephalogram signal X and some of RMS of the artifact component signals C1 to C5 of the electroencephalogram signal X. More specifically, SQI of each of the electroencephalogram signals X is calculated based on the following formula (2). SQI is shown in percentage (%). The higher a value of SQI of the electroencephalogram signal X, the higher the signal quality of the electroencephalogram signal X. Thus, the signal quality of each of electroencephalogram signals can be objectively evaluated through the value of SQI.

$$\\ \begin{matrix}S Q I ( \% ) = \frac{{RMS}_{X} - ( {RMS}_{C 5} + {RMS}_{C 2} + {RMS}_{C 1} )}{{RMS}_{X}} \times 100 \% & ( 2 )\end{matrix}$$

Here, RMSₓ indicates RMS of the electroencephalogram signal X. RMS_{C5} indicates RMS of the artifact component signal C5 caused by the electromyogram. RMS_{-C2} indicates RMS of the artifact component signal C2 caused by the electrode lead wire. RMS_{c1} indicates RMS of the artifact component signal C1 caused by the attachment failure of the electrode. Thus, SQI of each of the electroencephalogram signals X is calculated based on RMSₓ of each electroencephalogram signal X and RMS (RMS_{C1}, RMS_{C2}, RMS_{C5}) of the artifact component signals C1, C2, and C5 of each of the electroencephalogram signals X. For example, SQI of the electroencephalogram signal X_{Fp1} of the electrode Fp1 is calculated based on RMS of the electroencephalogram signal X_{Fp1} and RMS of the artifact component signals C1, C2, and C5 of the electroencephalogram signal X_{Fp1}. In the present embodiment, RMS of the artifact component signals C3 and C4 are not used in the calculation of the SQI, but these RMS may be used in the SQI calculation.

Next, in step S5, the controller 2 is configured to determine a comprehensive SQI of the plurality of electroencephalogram signals X, based on SQI of electroencephalogram signals X. Here, the comprehensive SQI indicates a comprehensive signal quality of the plurality of electroencephalogram signals X. In this respect, in the present embodiment, the controller 2 is configured to determine a minimum value among SQI of the electroencephalogram signals X as the comprehensive SQL. A representative value such as an average value or a median value of each electroencephalogram signal X may be determined as the comprehensive SQI.

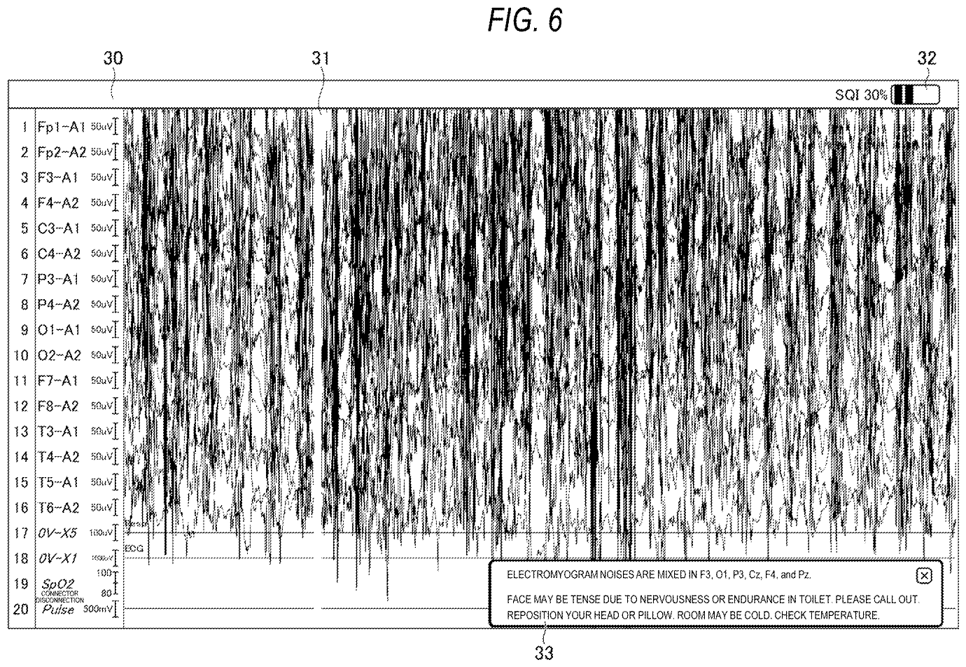

After the controller 2 determines the comprehensive SQI, the controller 2 may be configured to change a display content of an SQI indicator 32 indicating the value of the SQI on an electroencephalogram display screen 30 illustrated in FIG. 6. The controller 2 may be configured to update the display content of the SQI indicator 32 every time a value of the comprehensive SQI is updated for each predetermined period. Further, the controller 2 is configured to cause the display 4 to display an electroencephalogram display screen 30. As illustrated in FIG. 6, the electroencephalogram display screen 30 includes an electroencephalogram display area 31 in which electroencephalograms of channels (16 channels in this example) are displayed. An electroencephalogram signal of a channel 1 shown in the electroencephalogram display area 31 is an electroencephalogram signal corresponding to a difference between the electroencephalogram signal X_{Fp1} of the electrode Fp1 and the electroencephalogram signal X_{A1} of the electrode A1. An electroencephalogram signal of a channel 2 is an electroencephalogram signal corresponding to a difference between the electroencephalogram signal X_{Fp2} of the electrode Fp2 and the electroencephalogram signal X_{A2} of the electrode A2.

Next, in step S6, the controller 2 is configured to determine whether the comprehensive SQI is 50% or less. In a case where a determination result of step S6 is NO, the processing returns to step S1. On the other hand, in a case where the comprehensive SQI is 50% or less (the determination result of step S6 is YES), the controller 2 is configured to determine whether RMS of each of the artifact component signals C1 to C5 of each of the electroencephalogram signals X (electroencephalogram signals X_{Fp1} to X_{A2}) is greater than a threshold value (step S7). In this example, the controller 2 is configured to determine whether the comprehensive SQI is 50% or less, but in step S6, the controller 2 may be configured to determine whether the comprehensive SQI is X % (X is any value other than 50) or less.

In step S7, the controller 2 is configured to determine whether RMS_{C1} of the artifact component signal C1 of the electroencephalogram signal X is greater than a threshold value RMSₜₕ₁ associated with RMS_{C1}. Further, the controller 2 is configured to determine whether the RMS_{C2} of the artifact component signal C2 of the electroencephalogram signal X is greater than a threshold value RMSₜₕ₂ associated with RMS_{C2}. The controller 2 is configured to determine whether RMS_{C3} of the artifact component signal C3 of the electroencephalogram signal X is greater than a threshold value RMSₜₕ₃ associated with RMS_{C3}. Furthermore, the controller 2 is configured to determine whether RMS_{C4} of the artifact component signal C4 of the electroencephalogram signal X is greater than a threshold value RMSₜₕ₄ associated with RMS_{C4}. The controller 2 is configured to determine whether RMS_{C5} of the artifact component signal C5 of the electroencephalogram signal X is greater than a threshold value RMSₜₕ₅ associated with RMS_{C5}.

The processing of determining whether RMS of each of the artifact component signals C1 to C5 is greater than the respective one of the threshold values RMSₜₕ₁ to RMSₜₕ₅ is performed on each of the electroencephalogram signals X_{Fp1} to X_{A2}. Therefore, the threshold value determination processing related to RMS of the artifact component signals C1 to C5 is performed for each of the 21 types of electroencephalogram signals X_{Fp1} to X_{A2}. That is, 105 times, which is that 21 multiplied by 5, of threshold value determination processing are executed in step S6.

For example, in a case where RMS_{C1} of the artifact component signal C1 of the electroencephalogram signal X_{Fp1} is greater than the threshold value RMSₜₕ₁, the controller 2 is configured to determine that the artifact caused by the attachment failure of the electrode is mixed in the electroencephalogram signal X_{Fp1}. In a case where RMS_{C2} of the artifact component signal C2 of the electroencephalogram signal X_{Fp1} is greater than the threshold value RMSₜₕ₂, the controller 2 is configured to determine that the artifact (AC noise) caused by the electrode lead wire is mixed in the electroencephalogram signal X_{Fp1}. In a case where RMS_{C3} of the artifact component signal C3 of the electroencephalogram signal X_{Fp1} is greater than the threshold value RMSₜₕ₃, the controller 2 is configured to determine that the artifact (electro cardiogram noise) caused by the electro cardiogram is mixed in the electroencephalogram signal X_{Fp1}. In a case where RMS_{C4} of the artifact component signal C4 of the electroencephalogram signal X_{Fp1} is greater than the threshold value RMSₜₕ₄, the controller 2 is configured to determine that the artifact (eye movement noise) caused by the eye movement is mixed in the electroencephalogram signal X_{Fp1}. In a case where RMS_{C5} of the artifact component signal C5 of the electroencephalogram signal X_{Fp1} is greater than the threshold value RMSₜₕ₅, the controller 2 is configured to determine that the artifact (electromyogram noise) caused by the electromyogram is mixed in the electroencephalogram signal X_{Fp1}.

In step S8, the controller 2 is configured to visually display information related to the artifact associated with the artifact component signal of which RMS is greater than the threshold value. In this respect, the controller 2 may be configured to cause the electroencephalogram display area 31 of the electroencephalogram display screen 30 to display artifact related information 33 indicating information related to the artifact.

The artifact related information 33 includes information indicating the type of artifact, information indicating an electrode associated with an electroencephalogram signal in which the artifact is mixed, and information indicating a measure for reducing the artifact. For example, in a determination result of step S7, RMS_{C5} of the artifact component signal C5 of the electroencephalogram signal X associated with each of the electrodes F3, O1, P3, Cz, F4, and Pz is greater than RMSₜₕ₅. In such a case, as illustrated in FIG. 6, the information indicating the artifact (that is, the electromyogram noise) associated with the artifact component signal C5 is displayed in the artifact related information 33. Further, information indicating the electrodes F3, O1, P3, Cz, F4, and Pz associated with the electroencephalogram signals in which the artifact is mixed is displayed in the artifact related information 33. Furthermore, information indicating a measure for reducing the electromyogram noise is displayed in the artifact related information 33.

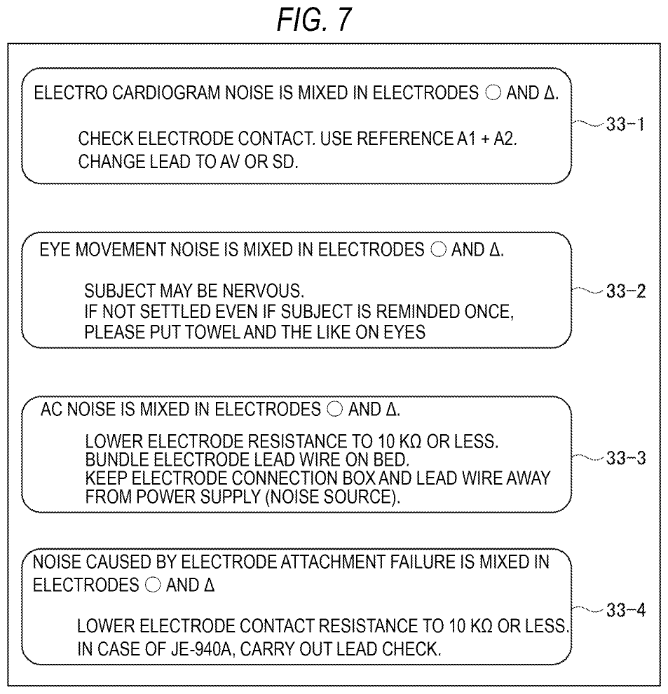

As illustrated in FIG. 7, artifact related information 33-1 to 33-4 may be displayed in the electroencephalogram display area 31 of the electroencephalogram display screen 30, according to the type of artifact. In a case where RMS_{C3} of the artifact component signal C3 of the electroencephalogram signal X related to a predetermined electrode is greater than RMSₜₕ₃, information indicating the electro cardiogram noise, information indicating an electrode associated with an electroencephalogram signal in which the electro cardiogram noise is mixed, and information indicating a measure for reducing the electro cardiogram noise are displayed in the artifact related information 33-1. In a case where RMS_{C4} of the artifact component signal C4 of the electroencephalogram signal X related to a predetermined electrode is greater than RMSₜₕ₄, information indicating the eye movement noise, information indicating an electrode associated with an electroencephalogram signal in which the eye movement noise is mixed, and information indicating a measure for removing the eye movement noise are displayed in the artifact related information 33-2.

In a case where RMS_{C2} of the artifact component signal C2 of the electroencephalogram signal X related to a predetermined electrode is greater than RMSₜₕ₂, information indicating the AC noise, information indicating an electrode associated with an electroencephalogram signal in which the AC noise is mixed, and information indicating a measure for removing the AC noise are displayed in the artifact related information 33-3. In a case where RMS_{C1} of the artifact component signal C1 of the electroencephalogram signal X related to the predetermined electrode is greater than RMSₜₕ₁, information indicating a noise (electrode attachment failure noise) caused by the electrode attachment failure, information indicating an electrode associated with an electroencephalogram signal in which the electrode attachment failure noise is mixed, and information indicating a measure for reducing the electrode attachment failure noise are displayed in the artifact related information 33-4.

In the example shown in FIG. 6, information related to one of the five types of artifacts is displayed in the electroencephalogram display area 31 as the artifact related information. In this respect, in the determination result of step S7, it is determined that a plurality of types of artifacts are mixed in the electroencephalogram signal X, information related to one of the plurality of types of artifacts mixed in the electroencephalogram signal X may be displayed as the artifact related information. In the present embodiment, the artifact caused by the factor other than the living body has a higher priority than the artifact caused by the living body. That is, in a case where at least one of the electroencephalogram signals X_{Fp1} to X_{A2} is mixed with the artifact caused by the factor other than the living body and the artifact caused by the living body, since removal of the artifact caused by the factor other than the living body is relatively easier than removal of the artifact caused by the living body, information related to the artifact caused by the factor other than the living body is first displayed in the electroencephalogram display area 31. In this manner, the medical worker is first leaded to reduce the artifact caused by the factor other than the living body from the electroencephalogram signal. In this case, as a result of an artifact reduction measure taken by the medical worker, after the artifact caused by the factor other than the living body is reduced, information related to the artifact caused by the living body is sequentially displayed in the electroencephalogram display area 31.

In particular, among artifacts caused by factors other than the living body, the artifact (electrode attachment failure noise) caused by the attachment failure of the electrode may have a higher priority than the artifact (AC noise) caused by the electrode lead wire. Further, among the artifacts caused by the living body, the artifact (electromyogram noise) caused by the electromyogram may have a highest priority, and the artifact (electro cardiogram noise) caused by the electro cardiogram may have a higher priority than the artifact (eye movement noise) caused by the eye movement. That is, a priority order of information display related to each artifact is as follows. Electrode attachment failure noise>AC noise>electromyogram noise>electro cardiogram noise>eye movement noise. For example, in a case in which five noises are simultaneously mixed in the electroencephalogram signals X_{Fp1} to X_{A2}, information related to the electrode attachment failure noise is first displayed, and after the noise is reduced by the medical worker through the measure against the artifact, information related to the AC noise is displayed.

The priority order of the information display related to each artifact is determined in consideration of difficulty of reducing the artifacts and of artifacts that greatly affect the electroencephalogram waveform. For example, since difficulty of reducing the artifact caused by the factor other than the living body is lower than difficulty of reducing the artifact caused by the living body, information related to the artifact caused by the factor other than the living body is preferentially displayed. Furthermore, since an influence of the electrode attachment failure noise on the electroencephalogram waveform is larger than an influence of the AC noise on the electroencephalogram waveform, display of the information related to the electrode attachment failure noise has a higher priority than display of the information related to the AC noise. The priority order of the information display related to each artifact is not particularly limited. The priority order may be appropriately set by the medical worker.

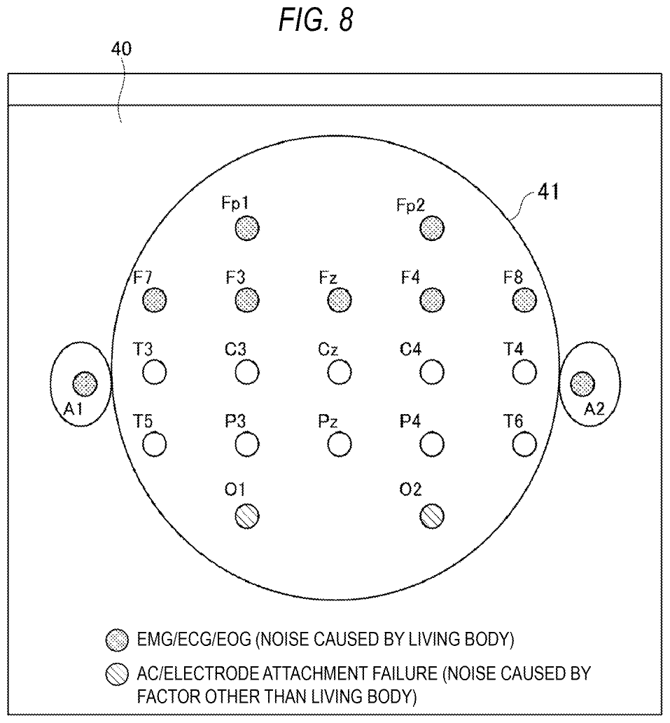

As illustrated in FIG. 8, information indicating the type of artifact mixed in each of the electroencephalogram signals may be visually presented in an electrode image 41 displayed on an electrode display screen 40. FIG. 8 is a diagram illustrating an example of the electrode display screen 40 on which the electrode image 41 indicating the type of artifact mixed in the electroencephalogram signal of each of the electrodes Fp1 to A2 is displayed. As illustrated in FIG. 8, in the electrode image 41, illustrations of the 21 types of electrodes Fp1 to A2 attached to the subject P are schematically displayed. Further, the information indicating the type of artifact mixed in each of the electroencephalogram signals is associated with a corresponding one of the electrodes Fp1 to A2.

Specifically, in the example illustrated in FIG. 8, in a case where the artifacts (electrode attachment failure noise and AC noise) caused by the factors other than the living body are mixed in the electroencephalogram signals X_{O1} and X_{O2} related to the electrodes O1 and O2, information indicating that the artifacts caused by the factors other than the living body are mixed in the electroencephalogram signals X_{O1} and X_{O2} of the electrodes O1 and O2 is displayed on the electrode image 41. More specifically, illustrations of the electrodes O1 and O2 are colored in a first color in a state in which the artifacts caused by the factors other than the living body are associated with the first color.

Furthermore, in a case where the artifacts (electromyogram noise, electro cardiogram noise, and eye movement noise) caused by the living body are mixed in the electroencephalogram signals X related to the electrodes Fp1, Fp2, F7, F3, Fz, F4, F8, A1, and A2, information indicating that the artifacts caused by the living body are mixed in the electroencephalogram signals X of the electrodes Fp1, Fp2, F7, F3, Fz, F4, F8, A1, and A2 is displayed on the electrode image 41. More specifically, illustrations of the electrodes Fp1, Fp2, F7, F3, Fz, F4, F8, A1, and A2 are colored in a second color in a state in which the artifacts caused by the living body are associated with the second color different from the first color.

In a case where no artifact is mixed in the electroencephalogram signals X related to the electrodes T3, C3, Cz, C4, T4, T5, P3, Pz, P4, and T6, illustrations of the electrodes T3, C3, Cz, C4, T4, T5, P3, Pz, P4, and T6 are not colored. In this manner, the medical worker can intuitively grasp the type of artifact mixed in the electroencephalogram signal of each electrode by visually recognizing the illustration of each electrode displayed on the electrode image 41.

In the example illustrated in FIG. 8, in a case where the plurality of artifacts are mixed in the electroencephalogram signal of the predetermined electrode, coloring of an illustration of the predetermined electrode is determined according to the priority order of the various artifacts. For example, when both the artifact caused by the living body and the artifact caused by the factor other than the living body are mixed in the electroencephalogram signal X of the electrode F7, since the artifact caused by the factor other than the living body has a higher priority than the artifact caused by the living body, the illustration of the electrode F7 is colored in the first color.

The electrode display screen 40 illustrated in FIG. 8 and the electroencephalogram display screen 30 illustrated in FIG. 6 may be simultaneously displayed on a display screen of the display 4. In this case, the medical worker can appropriately take a measure for removing the artifacts mixed in each electroencephalogram signal by simultaneously viewing the electroencephalogram display screen 30 and the electrode display screen 40 simultaneously displayed on the display 4.

In this manner, the controller 2 is configured to repeatedly execute a series of processing of steps S1 to S8 shown in FIG. 3. As described above, the processing of steps S2 to S8 may be repeatedly executed every time the electroencephalogram signals in the predetermined period (for example, a 2-second epoch) are obtained from the electrodes Fp1 to A2 in step S1.

According to the present embodiment, the controller 2 is configured to determine whether RMS of each of the artifact component signals C1 to C5 of each of the electroencephalogram signals X satisfies the threshold value, and the type of artifact mixed in the electroencephalogram signal X is specified. Thereafter, the artifact related information (particularly, information indicating the type of artifact and information indicating the measure for reducing the artifact) related to the artifact mixed in each of the electroencephalogram signals X is visually presented to the medical worker. In this way, it is possible to take the measure for reducing the artifact from the electroencephalogram signal through the artifact related information even for a medical worker who has little experience in electroencephalogram measurement. As a result, the medical worker can record an electroencephalogram signal having a high signal quality which is less contaminated with the artifact.

In the electrode image 41 illustrated in FIG. 7, information indicating the type of artifact mixed in the each electroencephalogram signal X is visually displayed in association with a respective one of the plurality of electrodes Fp1 to A2. In particular, a display color of the illustration of each of the electrodes Fp1 to A2 is set according to the type of artifact. As described above, even the medical worker who has little experience in the electroencephalogram measurement can appropriately take a measure to remove the artifact from each electroencephalogram signal by visually recognizing the electrode image 41 (in particular, the display color of the illustration of each electrode).

Further, according to the present embodiment, in a case where the comprehensive SQI is 50% (an example of a predetermined threshold value) or less, it is determined whether RMS (an example of the effective value) of each of the artifact component signals C1 to C5 of each electroencephalogram signal X satisfies the predetermined threshold value. As described above, only in the case in which the comprehensive SQI is low and a probability that the electroencephalogram signal X is mixed with the artifact is extremely high, a determination processing related to RMS of the artifact component signals C1 to C5 is executed. Accordingly, it is possible to reduce a calculation load and power consumption of a computer configured to execute the determination processing.

In the present embodiment, it is determined whether RMS of each artifact component signal of each electroencephalogram signal is greater than the threshold value in a case where the comprehensive SQI of the electroencephalogram signal X is 50% or less in step S6, but the present embodiment is not limited thereto. For example, the processing of steps S4 to S6 may not be executed in the series of processing of FIG. 2. In this case, the processing of step S7 may be executed after the processing of step S3 is executed.

In order to achieve the processing apparatus 1 according to the present embodiment by software, the physiological information processing program may be incorporated in the storage device 3 or the ROM in advance. Alternatively, the physiological information processing program may be stored in a computer readable storage medium such as a magnetic disk (for example, HDD and a floppy disk), an optical disk (for example, CD-ROM, DVD-ROM, and Blu-ray (registered trademark) disk), a magneto optical disk (for example, MO), a flash memory (for example, a SD card, a USB memory, and SSD). In this case, the physiological information processing program stored in the storage medium may be incorporated in the storage device 3. Further, after the program incorporated in the storage device 3 is loaded onto RAM, the processor may be configured to execute the program loaded on RAM. As described above, the physiological information processing method according to the present embodiment is executed by the processing apparatus 1.

The physiological information processing program may be downloaded from a computer on a communication network via the communication unit 5. In this case, the downloaded program may be incorporated in the storage device 3.

Although the embodiments of the presently disclosed subject matter have been described above, the technical scope of the presently disclosed subject matter should not be construed as being limited to the description of the present embodiments. The present embodiments are merely an example, and it is understood by those skilled in the art that various modifications of the embodiments are possible within the scope of the disclosed subject matters described in the claims. The technical scope of the presently disclosed subject matter should be determined based on the scope of the disclosed subject matters described in the claims and equivalents thereof

What is claimed is:

1. A physiological information processing method executed by a computer, the physiological information processing method comprising:
obtaining a plurality of electroencephalogram signals indicating an electroencephalogram of a subject, using a plurality of electrodes attached to a head of the subject, each of the plurality of electroencephalogram signals being associated with a respective one of the plurality of electrodes;
obtaining, from each of the plurality of electroencephalogram signals, a plurality of artifact component signals indicating different types of artifacts;
calculating effective values of the plurality of artifact component signals of each of the electroencephalogram signals;
determining whether the effective value of each of the artifact component signals of the electroencephalogram signal satisfies a predetermined condition; and
visually presenting information related to the artifact associated with the artifact component signal of which the effective value satisfies the predetermined condition.

2. The physiological information processing method according to claim 1,
wherein the information related to the artifact includes:
information indicating a type of the artifact; and
information indicating a measure for reducing the artifact.

3. The physiological information processing method according to claim 1,
wherein in a case where at least one of the effective values of the plurality of artifact component signals in each electroencephalogram signal satisfies the predetermined condition, information indicating a type of artifact mixed in each electroencephalogram signal is visually presented in association with a respective one of the plurality of electrodes.

4. The physiological information processing method according to claim 1,
wherein the plurality of electrodes include a first electrode,
the plurality of electroencephalogram signals include a first electroencephalogram signal associated with the first electrode,
the plurality of artifact component signals include a first artifact component signal indicating a first artifact, and
in a case where an effective value of the first artifact component signal associated with the first electroencephalogram signal is greater than a predetermined threshold value associated with the effective value, information related to the first artifact mixed in the first electroencephalogram signal is visually presented.

5. The physiological information processing method according to claim 4,
wherein the information related to the first artifact mixed in the first electroencephalogram signal includes:
information indicating a type of the first artifact; and
information indicating a measure for reducing the first artifact.

6. The physiological information processing method according to claim 1,
wherein the plurality of electrodes include a first electrode,
the plurality of electroencephalogram signals include a first electroencephalogram signal associated with the first electrode,
the plurality of artifact component signals include:
a first artifact component signal indicating a first artifact caused by a living body; and
a second artifact component signal indicating a second artifact caused by a factor other than the living body, and
in a case where a first effective value of the first artifact component signal associated with the first electroencephalogram signal is greater than a first threshold value associated with the first effective value, and a second effective value of the second artifact component signal associated with the first electroencephalogram signal is greater than a second threshold value associated with the second effective value, information associated with the second artifact mixed in the first electroencephalogram signal is visually presented prior to information associated with the first artifact mixed in the first electroencephalogram signal.

7. The physiological information processing method according to claim 1, further comprising:
calculating a plurality of signal quality indexes each indicating a signal quality of a respective one of the plurality of electroencephalogram signals, based on effective values of at least one of the plurality of artifact component signals of each electroencephalogram signal.

8. The physiological information processing method according to claim 7, further comprising:
determining a comprehensive signal quality index indicating a comprehensive signal quality of the plurality of electroencephalogram signals, based on the plurality of signal quality indexes,
wherein in a case where the comprehensive signal quality index is equal to or less than a predetermined threshold value, the determining whether the effective value of each of the artifact component signals of the electroencephalogram signal satisfies the predetermined condition is executed.

9. The physiological information processing method according to claim 1,
wherein the plurality of artifact component signals include:
a first artifact component signal indicating a first artifact caused by a living body; and
a second artifact component signal indicating a second artifact caused by a factor other than the living body, and
the second artifact includes:
an artifact caused by attachment failure of the electrode attached to the subject; and
an artifact caused by a lead wire connected to the electrode.

10. A non-transitory computer-readable storage medium storing a program comprising instructions which, when the program is executed by the computer, cause the computer to execute the physiological information processing method according to claim 1.

11. A physiological information processing apparatus comprising:
one or more processors; and
one or more memories configured to store a computer readable instruction,
wherein in a case where the computer readable instruction is executed by the one or more processors, the physiological information processing apparatus is configured to:
obtain a plurality of electroencephalogram signals indicating an electroencephalogram of a subject, using a plurality of electrodes attached to a head of the subject, each of the plurality of electroencephalogram signals being associated with a respective one of the plurality of electrodes,
obtain, from each of the plurality of electroencephalogram signals, a plurality of artifact component signals indicating different types of artifacts,
calculate effective values of the plurality of artifact component signals of each of the electroencephalogram signals,
determine whether the effective value of each of the artifact component signals of the electroencephalogram signal satisfies a predetermined condition, and
visually present information related to the artifact associated with the artifact component signal of which the effective value satisfies the predetermined condition.

12. The physiological information processing apparatus according to claim 11,
wherein the information related to the artifact includes:
information indicating a type of the artifact; and
information indicating a measure for removing the artifact.

13. The physiological information processing apparatus according to claim 11,
wherein in a case where at least one of the effective values of the plurality of artifact component signals in each electroencephalogram signal satisfies the predetermined condition, information indicating a type of artifact mixed in each electroencephalogram signal is visually presented.

14. The physiological information processing apparatus according to claim 11,
wherein in a case where the computer readable instruction is executed by the one or more processors, the physiological information processing apparatus is further configured to:
calculate a plurality of signal quality indexes each indicating a signal quality of a respective one of the plurality of electroencephalogram signals, based on effective values of at least one of the plurality of artifact component signals of each electroencephalogram signal;
determine a comprehensive signal quality index indicating a comprehensive signal quality of the plurality of electroencephalogram signals, based on the plurality of signal quality indexes; and
in a case where the comprehensive signal quality index is equal to or less than a predetermined threshold value, determine whether the effective value of each of the artifact component signals of the electroencephalogram signal satisfies the predetermined condition.
